(19) United States
(12) Patent Application Publication
(10) Pub. No.: US 2026/0116819 A1
(43) Pub. Date: Apr. 30, 2026

(54) PARTICLE SORTING METHOD, QUALITY EVALUATION METHOD, AND BURNING PROCESS CONTROL METHOD

(71) Applicant: TAIHEIYO CEMENT CORPORATION, Tokyo (JP)

(72) Inventors: Tatsuto HAJI, Tokyo (JP); Tomomi SATO, Tokyo (JP); Maiko YAMAGUCHI, Tokyo (JP); Daisuke KUROKAWA, Tokyo (JP)

(21) Appl. No.: 19/163,793

(22) PCT Filed: Mar. 22, 2024
(86) PCT No.: PCT/JP2024/011238

§ 371 Date: Sep. 9, 2025

(30) Foreign Application Priority Data
Mar. 28, 2023 (JP) 2023-050917

(51) Int. Cl. C04B 7/36; B07C 5/342; C04B 7/44; F27B 15/18; G05B 13/02; G06T 7/00; G06V 10/764; G06V 10/774; G06V 10/82; G06V 20/69

(52) U.S. Cl. CPC C04B 7/361; B07C 5/3425; C04B 7/44; F27B 15/18; G05B 13/0265; G06T 7/001; G06V 10/764; G06V 10/774; G06V 20/698; F27M 2003/03; G06T 2207/20081; G06T 2207/30132; G06V 10/82; G06V 2201/06

(57) ABSTRACT

A particle sorting method may more quickly and more reliably obtain image data in which particles suitable for evaluation has been imaged. The particle sorting method includes: a process (a) of collecting, as a particle sample, some of particles contained in a pulverized clinker or a cement particle group, and mixing the particle sample with a predetermined solvent to prepare a suspension; a process (b) of pouring the suspension into a flow path that has been predetermined, and imaging the suspension flowing through the flow path to obtain sorting image data; and a process (c) of applying the sorting image data to a first learned model, and sorting the sorting image data according to a type of an imaged particle, the first learned model being generated by performing machine learning based on first training input data is associated with a feature parameter serving as a reference.

### TECHNICAL FIELD

The present invention relates to a particle sorting method, a quality evaluation method, and a burning process control method, and in particular, a method for sorting particles contained in a pulverized clinker or a cement particle group, a quality evaluation method of a clinker or cement, and a burning process control method of a clinker.

### BACKGROUND ART

Cement is produced by mixing and pulverizing a clinker produced by burning a mixture of limestone, clay, silica stone, and an iron oxide raw material under predetermined burning conditions by using a rotary kiln, together with gypsum and a predetermined small amount of mixed material. Here, it is known that the quality of cement depends on burning conditions in a burning process using the rotary kiln. For example, in a case where burning conditions in a burning process are inappropriate, there can be a situation in which expected strength fails to be obtained in concrete produced by using cement that has been produced under the burning conditions.

In view of the circumstances described above, in order to prepare cement of a desired quality level, conventionally, in a burning process of a clinker, a portion of a produced clinker is collected as a sample, and burning conditions are adjusted on the basis of structural observation, component analysis, or the like. As a method for adjusting the burning conditions of the clinker, for example, a method in which a person in charge of evaluation observes a specified crystal contained in a produced clinker, and adjusts a temperature rising rate in the burning process or a time period during which the high temperature is maintained on the basis of characteristics of the crystal, has been proposed (see Patent Document 1 described below).

### PRIOR ART DOCUMENTS

### Patent Document

Patent Document 1: JP-A-9-052741

### SUMMARY OF THE INVENTION

### Problems to be Solved by the Invention

The method described in Patent Document 1 described above is a method in which a person in charge of evaluation observes particles obtained by pulverizing a clinker, and adjusts burning conditions of the clinker on the basis of a shape or a size of a specified crystal. However, in a method in which a person in charge of evaluation observes an evaluation target, and evaluates the evaluation target on the basis of individual determination criteria, as described above, advanced knowledge and sufficient experience are required from selection of particles to be observed to quality evaluation.

Furthermore, in the method described in Patent Document 1 described above, it often takes an enormous amount of time to obtain a particle sample that is suitable as an evaluation target. Particles sample to be used for evaluation are collected from a clinker that has been pulverized by using a disk mill or the like, and even if an obtained amount is about several mg, an enormous number of particles are contained. Therefore, in the conventional method, there is also a circumstance where a significant effort and time are required to discover a desired crystal in a state suitable for evaluation from the sample.

In view of the problems described above, an object of the present invention is to provide a particle sorting method capable of more quickly and more reliably obtaining image data in which a particle suitable for evaluation has been imaged. Another object of the present invention is to provide a quality evaluation method and a burning process control method that do not vary among persons in charge.

### Means for Solving the Problems

A particle sorting method according to the present invention includes:
a process (a) of collecting, as a particle sample, some of particles contained in a pulverized clinker or a cement particle group, and mixing the particle sample with a predetermined solvent to prepare a suspension;
a process (b) of pouring the suspension into a flow path that has been predetermined, and imaging the suspension flowing through the flow path to obtain sorting image data; and
a process (c) of applying the sorting image data to a first learned model, and sorting the sorting image data according to a type of an imaged particle, the first learned model being generated by performing machine learning based on first training input data in which first training image data obtained by imaging a first reference particle extracted for a sorting reference from the pulverized clinker or the cement particle group is associated with a feature parameter serving as a reference for sorting the first reference particle.

Herein, “image data” is used with the intention of including still image data and moving image data. Furthermore, the moving image data is a set of pieces of image data that have been obtained at a predetermined frame rate, and the still image data can be obtained by extracting one frame of the moving image data. Therefore, the still image data and the moving image data are collectively referred to as “image data”.

The sorting image data is obtained by imaging the suspension that flows through the flow path by using a camera, a video camera, or the like, but the sorting image data may be obtained image data with no change, or may be image data obtained by extracting a partial region from the obtained image data.

Furthermore, herein, a “feature parameter” is a parameter relating to appearance characteristics of a particle, such as a shape, a size, a light transmittance, or a refractive index of the particle.

By imaging the suspension that flows through the flow path, it is possible to obtain a large amount of image data indicating particles contained in the suspension, that is, individual particles contained in the pulverized clinker or the cement particle group in a shorter time than is conventional. Then, the obtained large amount of image data is applied to the first learned model, and is automatically sorted.

Accordingly, according to the sorting method, it is possible to quickly obtain a large amount of image data in which particles contained in the particle group have been individually imaged. Moreover, in the sorting method, the obtained large amount of data can be automatically sorted on the basis of predetermined conditions, and desired image data can be extracted from the large amount of image data more quickly than is conventional.

In the particle sorting method,
the process (a) may be a process of mixing the particle sample with a solvent having a refractive index of 1.65 or more and 1.75 or less, and preparing the suspension. Note that, as the refractive index of the solvent, a value that has been measured by using a publicly known refractometer (for example, an Abbe refractometer) can be employed.

Moreover, in the particle sorting method,
the feature parameter of the first reference particle may include a feature parameter for sorting an alite crystal and a belite crystal, and
the process (c) may be a process of performing sorting to determine whether at least the sorting image data is image data in which the alite crystal or the belite crystal has been imaged.

Furthermore, in the particle sorting method,
the process (b) may be a process of obtaining the sorting image data by using a polarizing microscope.

Clinkers contain compounds such as tricalcium silicate (3CaO·SiO₂), which is also called alite, and dicalcium silicate (2CaO·SiO₂), which is also called belite. It is known that a state of particles after pulverization or a content percentage with respect to these compounds affects the speed of a hydration reaction, the time of strength development, or the like of cement. Therefore, a crystal of the compound contained in the pulverized clinker is imaged, and the shape or the size of the crystal is observed, and therefore the quality of cement can be evaluated.

From among the compounds, belite is a compound that exhibits hardness that is higher than that of alite, and therefore belite is likely to remain as a monophase crystal to which almost no other compounds are bonded in a state where the size of the pulverized clinker is maintained to some extent. Stated another way, belite is likely to be extracted as a sample particle in an ideal crystal state, and therefore it can be said that belite is a compound suitable for stable quality prediction.

Furthermore, it is known that belite contributes particularly to the long-term strength of cement, and the long-term strength of cement can be estimated by observing belite crystals contained in the pulverized clinker.

In addition, in a case where a compound such as alite or belite, as described above, is mixed with a solvent that is a liquid to prepare a suspension, the suspension has birefringence in which appearance of crystals in a turbid liquid changes depending on the solvent. Such a change in appearance is affected by a relationship between the refractive index of the solvent and the refractive index of the crystal.

The range described above of the refractive index of the solvent is a range of the refractive index of the solvent within which particularly an alite crystal and a belite crystal contained in the suspension can be easily checked.

Furthermore, a microscope can be used to more clearly image the shape of the particle, but it is preferable that a polarizing microscope be used in consideration of the birefringence of the suspension.

A quality evaluation method according to the present invention is
a quality evaluation method for evaluating quality of a clinker or cement, the quality evaluation method including:
the particle sorting method; and
a process (d) of evaluating the quality of the clinker or the cement on the basis of the sorting image data that has been sorted in the process (c).

In the quality evaluation method,
the process (d) may be a process of estimating and evaluating a strength of cement to be produced from a clinker or cement from which the particle sample has been collected on the basis of the feature parameter of the particle sample.

In the quality evaluation method,
the process (d) may be a process of applying the sorting image data that has been sorted in the process (c) to a second learned model, and evaluating the quality of the clinker or the cement, the second learned model being generated by performing machine learning based on second training input data in which second training image data obtained by imaging a second reference particle extracted for an evaluation reference from the pulverized clinker or the cement particle group is associated with quality related information of a clinker or the cement particle group from which the second reference particle has been obtained.

Herein, “quality-related information” is information relating to the quality of a clinker or cement. Specific examples thereof include fluidity, a calorific value at the time of a hydration reaction, setting, and strength development.

In a case where a skilled person necessarily performs evaluation, but in a case where persons in charge of evaluation that are different in manufacturing sites perform evaluation, the determination criteria vary depending on the persons in charge of evaluation, and there is a concern that the evaluation results differ. Accordingly, according to the evaluation method, the sorting of image data and evaluation based on the image data are automated, and therefore it is possible to evaluate the clinker or the cement more quickly and with less variation than is conventional.

A burning process control method according to the present invention is
a method for controlling a burning process of a clinker, the method including:
the particle sorting method; and
a process (e) of adjusting conditions of the burning process of the clinker on the basis of the sorting image data that has been sorted in the process (c).

In the burning process control method,
the process (e) may be a process of estimating and evaluating a strength of cement to be produced from a clinker or cement from which the particle sample has been collected on the basis of the feature parameter of the particle sample, and further adjusting the conditions of the burning process of the clinker on the basis of a result of estimation.

In the burning process control method,
the process (e) may be a process of applying the sorting image data that has been sorted in the process (c) to a second learned model to obtain a quality evaluation result of a clinker or cement, and adjust the conditions of the burning process of the clinker on the basis of the quality evaluation result, the second learned model being generated by performing machine learning based on second training input data in which second training image data obtained by imaging a second reference particle extracted for an evaluation reference from the pulverized clinker or the cement particle group is associated with quality related information of a clinker or cement from which the second reference particle has been obtained.

According to the control method, the conditions of the burning process are feedback-controlled every time the burning of the clinker is performed, and therefore the burning process of the clinker is appropriately adjusted to automatically converge on optimum conditions.

### Effect of the Invention

According to the present invention, a particle sorting method capable of more quickly and more reliably obtaining image data in which a particle suitable for evaluation has been imaged is achieved. Furthermore, a quality evaluation method and a burning process control method that do not vary among persons in charge are achieved.

### BRIEF DESCRIPTION OF THE DRAWINGS

FIG. 1 is a diagram schematically illustrating the entire configuration of a burning process control system of a clinker.

FIG. 2 is a diagram schematically illustrating a configuration of a particle sample device.

FIG. 3 is a diagram schematically illustrating a configuration of a control terminal.

FIG. 4 is a flowchart illustrating an example of the order of respective processes performed by the burning process control system of the clinker.

### MODE FOR CARRYING OUT THE INVENTION

Hereinafter, a particle sorting method, a quality evaluation method, and a burning process control method according to the present invention will be described with reference to the drawings. Note that each of the drawings described below is a drawing merely illustrating an example for explaining the particle sorting method, the quality evaluation method, and the burning process control method according to the present invention.

The particle sorting method according to the present invention is a method for imaging a sample collected from a pulverized clinker or a particle group of cement, obtaining pieces of sorting image data of individual particles, and automatically sorting the pieces of sorting image data by using a learned model.

Moreover, the quality evaluation method according to the present invention is a method for evaluating the quality of a clinker or cement on the basis of the pieces of sorting image data that have been sorted by using the particle sorting method described above. The burning process control method according to the present invention is a method for controlling a burning process of a clinker on the basis of the pieces of sorting image data that have been sorted by using the particle sorting method described above.

Note that the learned model to which the pieces of sorting image data are applied is a learned model generated by performing machine learning based on first training input data in which first training image data obtained by imaging a first reference particle extracted for a sorting reference from a pulverized clinker or a cement particle group is associated with a feature parameter serving as a reference for sorting the first reference particle, and the learned model corresponds to the first learned model.

Furthermore, the burning process control method according to the present invention may be a method for controlling a burning process of a clinker on the basis of a quality evaluation result obtained by using the quality evaluation method according to the present invention.

First, machine learning for generating a learned model to be used in the present invention will be described. Examples of a method of machine learning for generating the learned model to be used in the present invention include a neural network, linear regression, a decision tree, support vector regression, an ensemble method, a support vector machine, discriminant analysis, a naive Bayes method, and a nearest neighbor method. One type of these methods may be used alone, or two or more types thereof may be used in combination.

From among these methods, machine learning using the neural network is preferably selected from the viewpoint of being able to predict quality with higher accuracy. As the neural network, a hierarchical neural network having one or more intermediate layers between an input layer and an output layer is suitable from the viewpoint of being able to predict quality with higher accuracy.

Examples of the neural network include a convolutional neural network (CNN) such as a 3D convolutional neural network (3D CNN), a deep neural network (DNN), a recurrent neural network (RNN), a long short-term memory (LSTM) neural network (an improved recurrent neural network using the LSTM), and the like.

From among these neural networks, the 3D convolutional neural network (a neural network including a convolution layer, a pooling layer, or the like as an intermediate layer) that has performance that is excellent in the field of image recognition, and relates to the time axis is more suitable. The 3D convolutional neural network can detect a feature (including a feature that changes according to a temporal change) from plural pieces of image data obtained at different times, and can generate a second learned model that is capable of performing classification or regression by using the feature. In the convolutional neural network, the number of layers including a combination of the convolution layer and the pooling layer is preferably two or more, and more preferably, three or more, from the viewpoint of being able to perform prediction with higher accuracy.

Furthermore, as a tool for performing machine learning, for example, “TensorFlow (registered trademark)”, which is a software library developed by Google Inc., “IBM Watson (registered trademark)”, which is a system developed by IBM Corporation, or the like can be used.

Next, an embodiment of the burning process control method according to the present invention will be described by using a burning process control system 1 serving as an embodiment example for carrying out the method.

### [Burning Process Control System]

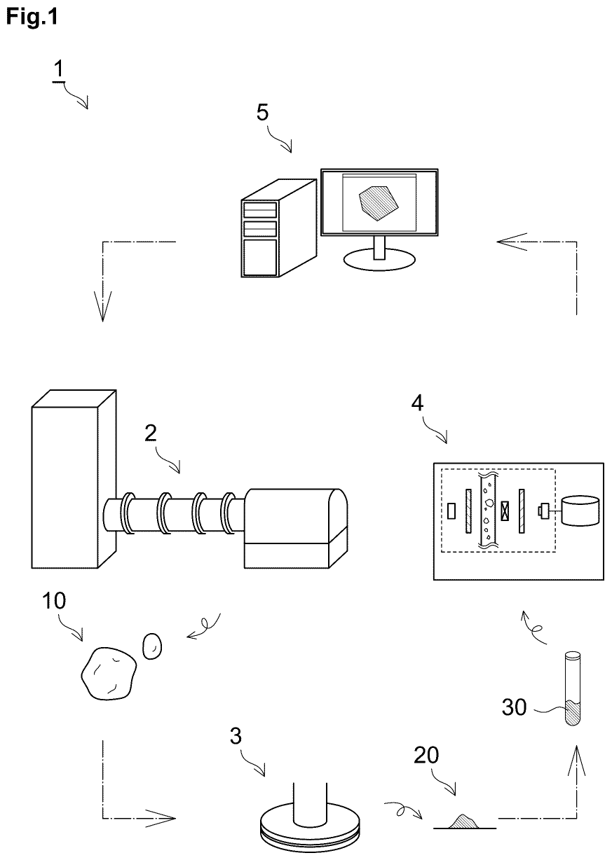

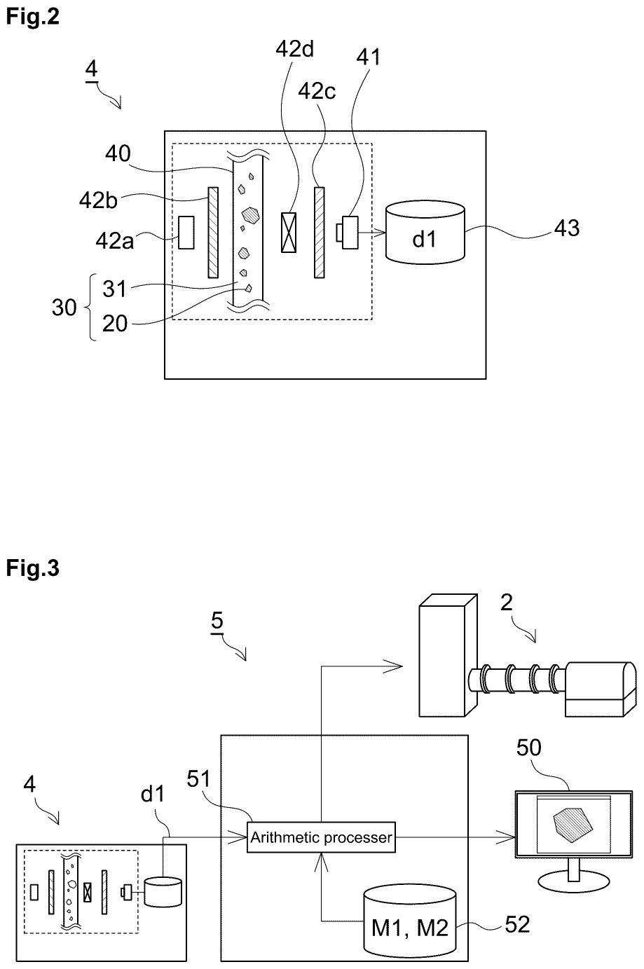

FIG. 1 is a diagram schematically illustrating the entire configuration of the burning process control system 1 of a clinker. Furthermore, FIG. 2 is a diagram schematically illustrating a configuration of a sample imaging device 4, and FIG. 3 is a diagram schematically illustrating a configuration of a control terminal 5. As illustrated in FIG. 1, the burning process control system 1 of a clinker according to the present embodiment includes a rotary kiln 2, a disk mill 3, the sample imaging device 4, and the control terminal 5.

The rotary kiln 2 burns a fed raw material on the basis of burning conditions that have been set by the control terminal 5, and produces a clinker 10.

The clinker 10 produced by the rotary kiln 2 is fed into the disk mill 3, and the disk mill 3 pulverizes the clinker 10.

A portion of the clinker 10 that has been pulverized into particles by the disk mill 3 is mixed as a particle sample 20 with a solvent 31. Suspension 30 serving as a mixed solution of the particle sample 20 and the solvent 31 is applied to the sample imaging device 4. Note that the suspension 30 is prepared, for example, by mixing the particle sample 20 of 1 mg and the solvent 31 of 1 ml.

Here, the solvent 31 according to the present embodiment is a liquid having a refractive index of 1.65 or more and 1.75 or less. A solvent having a desired refractive index can be obtained by mixing solvents such as bromoform or diiodomethane.

As illustrated in FIG. 2, the sample imaging device 4 includes a flow path 40 through which the suspension 30 flows inside, a camera 41 that images the suspension 30 that flows through the flow path 40, a light source 42a that is configured to secure luminance of an imaging field of view, a first polarizing plate 42b located between the light source 42a and the flow path 40 and a second polarizing plate 42c located between the camera 41 and an objective lens 42d, the first polarizing plate 42b and the second polarizing plate 42c being configured to enable birefringence to be observed, the objective lens 42d that is configured to enlarge and display the particle sample 20 inside the flow path 40, and a memory 43 that stores sorting image data d1 that has been obtained as a result of imaging performed by the camera 41. Here, in imaging, an arbitrary optical microscope having a structure in which the polarizing plates (42b and 42c) have been removed from the sample imaging device 4 may be used, but in view of an influence of the birefringence of the suspension 30, it is preferable that a polarizing microscope having a structure that is similar to that of the sample imaging device 4 be used.

In the sample imaging device 4, the camera 41 images the suspension 30 that flows through the flow path 40, and obtains the sorting image data d1 in which an individual particle sample 20 has been imaged. Then, the sorting image data d1 that has been obtained by the camera 41 is stored in the memory 43.

As the sample imaging device 4, for example, flow imaging microscope FlowCam 8100 (from Yokogawa Electric Corporation) can be employed.

Note that in the sample imaging device 4 according to the present embodiment, the suspension 30 is caused to flow through the flow path 40 at a flow rate of 0.03 ml/min, and about 100,000 pieces of sorting image data d1 are obtained from one sample of the suspension 30 in 20 minutes to 30 minutes.

As another method, in a case where a particle filter function is mounted on the sample imaging device 4, about 2,000 pieces of sorting image data d1 that have been sorted from one sample of the suspension 30, by using the same function may be obtained. The “particle filter function” described here refers to a function of setting a parameter, such as circularity or a diameter, of a particle to exclude, from a target to be stored, an image that does not meet a purpose from among a large number of images.

As illustrated in FIG. 3, the control terminal 5 includes a display 50, an arithmetic processer 51, and a storage 52. The storage 52 of the control terminal 5 stores a first learned model M1 and a second learned model M2. When the control terminal 5 has received an input of pieces of sorting image data d1 from the sample imaging device 4, the arithmetic processer 51 applies the pieces of sorting image data d1 to the first learned model M1.

Then, the arithmetic processer 51 applies sorted pieces of image data that have been output from the first learned model M1 to the second learned model M2. Thereafter, the arithmetic processer 51 adjusts the setting of the rotary kiln 2, on the basis of setting data that relates to the burning process of a clinker, and that has been output from the second learned model M2.

The first learned model M1 according to the present embodiment is a learned model generated by performing machine learning based on first training input data in which first training image data obtained by imaging a first reference particle extracted for a sorting reference from a pulverized clinker is associated with a feature parameter serving as a reference for sorting the first reference particle.

Stated another way, the first learned model M1 is a learned model that, when the sorting image data d1 has been applied, determines a type of an imaged particle, and automatically sorts the sorting image data.

The second learned model M2 according to the present embodiment is a learned model generated by performing machine learning based on second training input data in which second training image data obtained by imaging a second reference particle extracted for an evaluation reference from a pulverized clinker is associated with quality related information of the clinker from which the second reference particle has been obtained.

Stated another way, the second learned model M2 is a learned model that, when image data that has been sorted by the first learned model M1 has been applied, analyzes a feature parameter, such as a shape or a size, of an indicated particle, and outputs setting data of a burning process of a clinker.

Note that, as the first reference particle and the second reference particle, an arbitrary particle can be selected, and one or more types of particles can be selected. However, the first reference particle and the second reference particle according to the present embodiment are each an alite crystal and a belite crystal. Here, as the first reference particle and the second reference particle, it is preferable that a crystal in a state where damage and deformation do not occur and in a state where no other compounds are bound as much as possible be extracted.

Here, in a case where the alite crystal and the belite crystal are used as main targets to be imaged, the refractive index of the solvent 31 falls preferably within a range of 1.65 or more and 1.75 or less, and more preferably, within a range of 1.69 or more and 1.71 or less in such a way that the crystals can be more clearly imaged. However, it is sufficient if the refractive index of the solvent 31 falls within a range suitable for a crystal to be imaged, and the refractive index of the solvent 31 may be out of the range described above.

### [Burning Process Control Method]

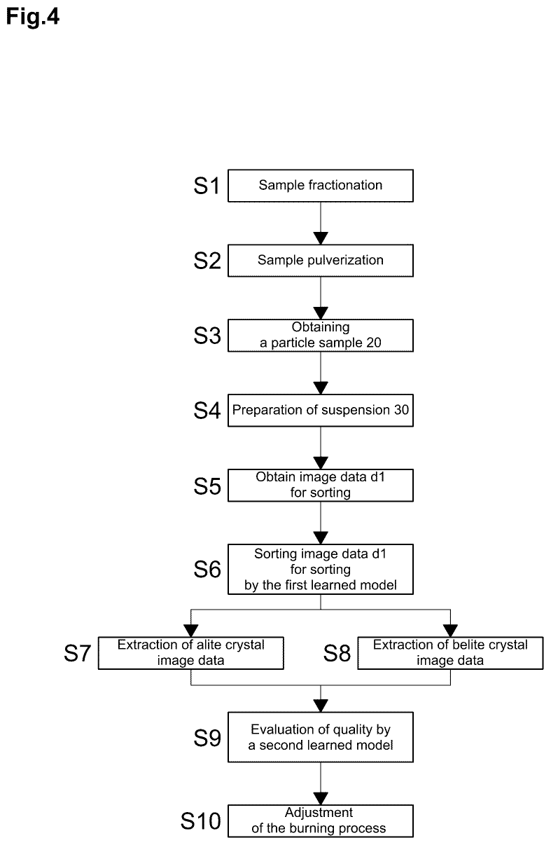

Next, the burning process control method according to the present invention will be described along the flowchart illustrated in FIG. 4.

First, a portion of the clinker 10 that has been produced by the rotary kiln 2 and has been extracted is fractionated as a sample (step S1).

The clinker 10 that has been fractionated as a sample is pulverized by the disk mill 3 (step S2).

A portion of the pulverized clinker 10 is obtained as the particle sample 20 (step S3).

The particle sample 20 and the solvent 31 are mixed to prepare the suspension 30 (step S4). Step S4 described above corresponds to the process (a).

The suspension 30 is applied to the sample imaging device 4, and pieces of sorting image data d1 are obtained (step S5). Step S5 described above corresponds to the process (b).

The pieces of sorting image data d1 that have been obtained by the sample imaging device 4 are input to the control terminal 5, are applied to the first learned model M1, and are sorted (step S6). Note that, in the present embodiment, extraction of image data indicating an alite crystal (step S7) and extraction of image data indicating a belite crystal (step S8) are performed on the pieces of sorting image data d1 by the first learned model M1. Step S6 to step S8 described above correspond to the process (c).

Pieces of image data that have been sorted into the image data indicating the alite crystal and the image data indicating the belite crystal are applied to the second learned model M2, and the quality of cement to be produced from the sample from which the particle sample 20 has been obtained is evaluated (step S9).

Note that in the present embodiment, the second learned model M2 estimates and evaluates the developed strength of concrete to be prepared from the cement, and derives the burning conditions of the burning process on the basis of a result of estimation and evaluation, but a characteristic to be evaluated and a characteristic from which the burning conditions are derived may be a characteristic other than the developed strength.

The second learned model M2 further outputs data relating to the burning conditions of the burning process on the basis of the result of estimation and evaluation. Then, the control terminal 5 adjusts burning conditions in the rotary kiln 2 on the basis of the data (step S10). A series of processes of step S9 and step S10 corresponds to the process (e).

After step S10 has been executed, the processes of step S1 to step S10 are repeated every time a process of producing the next clinker is performed.

By repeating the respective processes described above, burning conditions in the burning process of a clinker are automatically adjusted in such a way that the characteristics of a clinker to be produced approach desired characteristics. Stated another way, image data in which a particle that is suitable for evaluation has been imaged can be obtained more quickly and more reliably, and burning process control that does not vary among persons in charge is achieved.

The burning process control system 1 described above has been described as a system that performs the processes of step S1 to step S10, every time a clinker is produced, to control a burning process, but the burning process control system 1 described above can be used for quality evaluation of a produced clinker.

For example, the second learned model M2 may be a learned model that outputs data relating to the quality evaluation of the produced clinker, and the control terminal 5 may be configured to complete a series of processes by displaying a quality evaluation result on the display 50. In such a case, step S9 described above corresponds to the process (d).

Note that step S9 and step S10 described above may be performed to output the quality evaluation result or the control conditions of the burning process on the basis of a table that has been stored in advance in the storage 52 of the control terminal 5, and in which a feature parameter of a particle is associated with the quality information of a clinker, without using the second learned model M2.

Furthermore, the processes of step S4 to step S6 described above, namely, the particle sorting method, can be applied to not only the quality evaluation of a clinker or cement, and burning process control on the clinker, but also, for example, control on a process of mixing and pulverizing a clinker together with gypsum and a predetermined small amount of mixed material (a cement finishing process).

### EXAMPLES

Hereinafter, as an example, a result of sorting belite particles from a cement sample by using the method described above will be described. However, the present invention is not limited to this example.

As a powder sample 20, three different types of cement samples were prepared, and were assumed to be Level 1 to Level 3, respectively. Specifically, it was assumed that a powder sample 20 made of Ordinary Portland cement (from TAIHEIYO CEMENT CORPORATION) is Level 1, a powder sample 20 made of moderate heat cement (from TAIHEIYO CEMENT CORPORATION) is Level 2, and a powder sample 20 made of Low heat Portland cement (from TAIHEIYO CEMENT CORPORATION) is Level 3.

As the solvent 31, a mixed solution obtained by mixing bromoform (from FUJIFILM Wako Pure Chemical Corporation) and diiodomethane (from KANTO CHEMICAL CO., INC.) at a ratio of 1:3 was prepared. The refractive index of the obtained mixed solution was measured by using an Abbe refractometer (NAR-4T from ATAGO CO., LTD.), and was discovered to be 1.70.

For each of the levels, the powder sample 20 of 1 mg and the solvent 31 of 5 mL were mixed to prepare the suspension 30. The obtained suspension 30 was imaged by using a flow imaging microscope (FlowCam 8100 from Yokogawa Electric Corporation) while flowing at a flow rate of 0.03 mL/min for 30 minutes to obtain a large number of pieces of sorting image data d1.

The obtained pieces of sorting image data d1 were introduced into the arithmetic processer 51 in which the first learned model M1 was recorded, and a target belite particles were sorted. Results are indicated in Table 1. Note that a skilled operator checked all pieces of image data that have been extracted as belite by the arithmetic processer 51, and the “accuracy rate” in Table 1 corresponds to a ratio of the number of pieces of image data that were determined to be belite by the skilled operator relative to the number of extracted pieces of image data. The skilled operator described here is, for example, an “expert” described in Patent Document 1, and is an expert who has knowledge of mineral crystals, is excellent in visual sensation relating to microscopic observation, and has experience of an analyzer.

TABLE 1

| | | Number of | Number | |
|---|---|---|---|---|
| | | inputs of | of sorted | Accuracy |
| | | sorting image | belite images | rate |
| | Powder sample 20 | data d1 (Piece) | (Piece) | (%) |
| Level 1 | Ordinary Portland | 1767 | 4 | 100 |
| | cement | | | |
| Level 2 | Moderate heat | 1872 | 29 | 100 |
| | Portland cement | | | |
| Level 3 | Low heat Portland | 2256 | 48 | 100 |
| | cement | | | |

According to the results of Table 1, belite particles were able to be sorted from cement particles with extremely high accuracy, by using the method described above.

### DESCRIPTION OF REFERENCE SIGNS

1 Burning process control system
2 Rotary kiln
3 Disk mill
4 Sample imaging device
5 Control terminal
10 Clinker
20 Particle sample
30 Suspension
31 Solvent
40 Flow path
41 Camera
42a Light source
42b First polarized version
42c Second polarizing plate
42d Objective lens
43 Memory
50 Display
51 Arithmetic processer
52 Storage
M1 First learned model
M2 Second learned model

1. A particle sorting method comprising:
(a) collecting, as a particle sample, some of particles contained in a pulverized clinker or a cement particle group, and mixing the particle sample with a predetermined solvent to prepare a suspension;
(b) pouring the suspension into a flow path that has been predetermined, and imaging the suspension flowing through the flow path to obtain sorting image data; and
(c) applying the sorting image data to a first learned model, and sorting the sorting image data according to a type of an imaged particle, the first learned model being generated by performing machine learning based on first training input data in which first training image data obtained by imaging a first reference particle extracted for a sorting reference from the pulverized clinker or the cement particle group is associated with a feature parameter serving as a reference for sorting the first reference particle.

2. The particle sorting method according to claim 1, wherein in the step (a), the particle sample is mixed with a solvent having a refractive index of 1.65 or more and 1.75 or less, and the suspension is prepared.

3. The particle sorting method according to claim 2, wherein
the feature parameter of the first reference particle includes a feature parameter for sorting an alite crystal and a belite crystal, and
in the step (c), sorting is performed to determine whether at least the sorting image data is image data in which the alite crystal or the belite crystal has been imaged.

4. The particle sorting method according to claim 1, wherein in the step (b), the sorting image data is obtained by using a polarizing microscope.

5. A quality evaluation method for evaluating quality of a clinker or cement, the quality evaluation method comprising:
the particle sorting method according to claim 1; and
(d) evaluating the quality of the clinker or the cement on a basis of the sorting image data that has been sorted in the step (c).

6. The quality evaluation method according to claim 5, wherein in the step (d), a strength of cement to be produced from a clinker or cement from which the particle sample has been collected is estimated and evaluated on the basis of the feature parameter of the particle sample.

7. The quality evaluation method according to claim 5, wherein in the step (d), the sorting image data that has been sorted in the step (c) is applied to a second learned model, and the quality of the clinker or the cement is evaluated, the second learned model being generated by performing machine learning based on second training input data in which second training image data obtained by imaging a second reference particle extracted for an evaluation reference from the pulverized clinker or the cement particle group is associated with quality related information of a clinker or the cement particle group from which the second reference particle has been obtained.

8. A burning process control method for controlling a burning process of a clinker, the burning process control method comprising:
the particle sorting method according to claim 1; and
(e) adjusting conditions of the burning process of the clinker on a basis of the sorting image data that has been sorted in the step (c).

9. The burning process control method according to claim 8, wherein in the step (e), a strength of cement to be produced from a clinker or cement from which the particle sample has been collected is estimated and evaluated on the basis of the feature parameter of the particle sample, and the conditions of the burning process of the clinker are further adjusted on the basis of a result of estimation.

10. The burning process control method according to claim 8, wherein in the step (e), by applying the sorting image data that has been sorted in the step (c) to a second learned model, a quality evaluation result of a clinker or cement is obtained, and the conditions of the burning process of the clinker are adjusted on the basis of the quality evaluation result, the second learned model being generated by performing machine learning based on second training input data in which second training image data obtained by imaging a second reference particle extracted for an evaluation reference from the pulverized clinker or the cement particle group is associated with quality related information of a clinker or cement from which the second reference particle has been obtained.

11. A quality evaluation method for evaluating quality of a clinker or cement, the quality evaluation method comprising:
the particle sorting method according to claim 2; and
(d) evaluating the quality of the clinker or the cement on a basis of the sorting image data that has been sorted in the step (c).

12. A quality evaluation method for evaluating quality of a clinker or cement, the quality evaluation method comprising:
the particle sorting method according to claim 3; and
(d) evaluating the quality of the clinker or the cement on a basis of the sorting image data that has been sorted in the step (c).

13. A quality evaluation method for evaluating quality of a clinker or cement, the quality evaluation method comprising:
the particle sorting method according to claim 4; and
(d) evaluating the quality of the clinker or the cement on a basis of the sorting image data that has been sorted in the step (c).

14. The quality evaluation method according to claim 11, wherein in the step (d), a strength of cement to be produced from a clinker or cement from which the particle sample has been collected is estimated and evaluated on the basis of the feature parameter of the particle sample.

15. The quality evaluation method according to claim 11, wherein in the step (d), the sorting image data that has been sorted in the step (c) is applied to a second learned model, and the quality of the clinker or the cement is evaluated, the second learned model being generated by performing machine learning based on second training input data in which second training image data obtained by imaging a second reference particle extracted for an evaluation reference from the pulverized clinker or the cement particle group is associated with quality related information of a clinker or the cement particle group from which the second reference particle has been obtained.

16. A burning process control method for controlling a burning process of a clinker, the burning process control method comprising:
the particle sorting method according to claim 2; and
(e) adjusting conditions of the burning process of the clinker on a basis of the sorting image data that has been sorted in the step (c).

17. A burning process control method for controlling a burning process of a clinker, the burning process control method comprising:
the particle sorting method according to claim 3; and
(e) adjusting conditions of the burning process of the clinker on a basis of the sorting image data that has been sorted in the step (c).

18. A burning process control method for controlling a burning process of a clinker, the burning process control method comprising:
the particle sorting method according to claim 4; and
(e) adjusting conditions of the burning process of the clinker on a basis of the sorting image data that has been sorted in the step (c).

19. The burning process control method according to claim 16, wherein in the step (e), a strength of cement to be produced from a clinker or cement from which the particle sample has been collected is estimated and evaluated on the basis of the feature parameter of the particle sample, and the conditions of the burning process of the clinker are further adjusted on the basis of a result of estimation.

20. The burning process control method according to claim 16, wherein in the step (e), by applying the sorting image data that has been sorted in the step (c) to a second learned model, a quality evaluation result of a clinker or cement is obtained, and the conditions of the burning process of the clinker are adjusted on the basis of the quality evaluation result, the second learned model being generated by performing machine learning based on second training input data in which second training image data obtained by imaging a second reference particle extracted for an evaluation reference from the pulverized clinker or the cement particle group is associated with quality related information of a clinker or cement from which the second reference particle has been obtained.
